(19) United States
(12) Patent Application Publication
(10) Pub. No.: US 2002/0146400 A1
(43) Pub. Date: Oct. 10, 2002

(54) Composition for reducing plasma triglycerides, platelet aggregation, and oxidative capacity

(76) Inventor: Anthony H. Cincotta, Tiverton, RI (US)

Correspondence Address: Raymond J. Lillie Esq., c/o Carella, Byrne, Bain,Gilfillan,, Cecchi, Stewart & Olstein, 6 Becker Farm Road, Roseland, NJ, 07068, US

(21) Appl. No.: 09/754,957

(22) Filed: Jan. 5, 2001

Related U.S. Application Data
(60) Provisional application No. 60/175,176, filed on Jan. 7, 2000.

(51) Int. Cl. A61K 35/78; A61K 31/355; A61K 31/375; A61K 31/202; A61K 31/195; A61K 31/015

(52) U.S. Cl. 424/94.1; 424/766; 514/458; 514/474; 514/560; 514/561; 514/763

(57) ABSTRACT

A composition comprising at least one unsaturated fatty acid, such as an omega-3 fatty acid; pantethine; and an antioxidant selected from the group consisting of Vitamin C, Vitamin E, tocotrienol, at least one carotenoid, at least one flavenoid, coenzyme Q10, and grape seed extract. Such active ingredients may be encapsulated in an encapsulating medium to form microparticles, which may be suspended in an aqueous solution. Such a composition reduces plasma triglyceride levels, platelet hyperaggregation, endothelium dysfunction, and tissue oxidative capacity, and thus reduces the risk of cardiovascular disease.

This application claims priority based on, and is a continuation-in-part of provisional application Serial No. 60/175,176, filed Jan. 7, 2000.

This invention relates to a composition which reduces plasma triglyceride and cholesterol levels, acts as a systemic antioxidant, and reduces the risk of endothelium dysfunction and platelet hyperaggregation, thereby reducing the risk of cardiovascular disease. More particularly, this invention relates to a composition which includes at least one unsaturated fatty acid, pantethine, and an antioxidant such as Vitamin C, Vitamin E, tocotrienol, lycopene, pycnogenol, coenzyme Q10, or grape seed extract.

Atherosclerotic cardiovascular disease is a leading cause of death in the world. (Martin, Am. J. Pathol., Vol. 153, pgs. 1319-1320 (1998)). Research indicates that a complex interaction of multiple cellular and biochemical events participate in the development of cardiovascular disease. (Griffin, Proc. Nutr. Soc., Vol. 58, pgs. 163-169 (1999)). Within the myriad of identified biochemical events and factors contributing to cardiovascular disease, there are included hypertriglyceridemia, oxidized low-density lipoprotein (LDL), and platelet aggregation. It has been demonstrated in a variety of studies that hypertriglyceridemia (elevated plasma levels of triglycerides) is a risk factor for cardiovascular disease, particularly coronary artery disease. (Hokanson, et al., J. Cardiovasc. Risk, Vol. 3, pgs. 213-219 (1996); Stampfer, et al., JAMA, Vol. 11, pgs. 882-888 (1996); Patsch, et al., Arterioscler. Thromb., Vol. 12, pgs. 1336-1345 (1992)). In intervention studies wherein hypertriglyceridemia is reduced by the administration of pharmaceutical agents, the risk of cardiovascular disease also is reduced. (Ericsson, et al., Am. J. Cardiol., Vol. 80, pgs. 1125-1129 (1997); Ericsson, et al., Lancet, Vol. 347, pgs. 849-853 (1996); Rubins, et al., N. Engl. J. Med., Vol. 341, pgs. 410-418 (1999)). Plasma triglycerides contained within circulating very low density lipoprotein (VLDL) and low density lipoprotein (LDL) molecules potentiate cardiovascular disease by a variety of proposed mechanisms. (Reaven, et al., Circulation, Vol. 93, pgs. 1780-1783 (1996)).

Also, in addition to the increased amounts of triglycerides within plasma VLDL and LDL, the oxidation of plasma lipoproteins, particularly VLDL and LDL, renders these endogenous molecules more atherogenic. (Griffin, 1999; Holvoet, et al., Atherosclerosis, Vol. 137, Supp. S33-8 (1998); Holvoet, et al., FASEB J., Vol. 8, pgs. 1279-1284 (1994)). Therefore, reducing plasma triglyceride levels as well as the extent of oxidized plasma lipoprotein would be of therapeutic value in protecting the cardiovascular system from atherosclerotic disease and in improving the physiologic integrity of the cardiovasculature itself.

Another factor in the development of atherosclerotic disease is an increased incidence of circulating platelet aggregation, which potentiates thrombus development; i.e., clot formation (Aronow, Drugs Aging, Vol. 15, pgs. 91-101 (1999)).

Inasmuch as the above-mentioned adverse biochemical events interact with each other and with other factors to exacerbate cardiovascular disease, it would be of therapeutic benefit to ameliorate the above-mentioned events.

It is therefore an object of the present invention to provide a composition that can reduce hypertriglyceridemia, provide antioxidant activity, particularly antioxidant activity with respect to LDL, and reduce platelet aggregation hyperactivity.

In accordance with an aspect of the present invention, there is provided a composition comprising (a) at least one unsaturated fatty acid; and at least one of: (b) pantethine; and (c) at least one antioxidant selected from the group consisting of Vitamin C, Vitamin E, tocotrienol, carotenoids, flavenoids, coenzyme Q10, and grape seed extract.

In one embodiment, the at least one unsaturated fatty acid is selected from the group consisting of omega-3 fatty acids, α-linolenic acid, and oleic acid.

In another embodiment, the at least one unsaturated fatty acid is at least one omega-3 fatty acid. Omega-3 fatty acids which may be employed in the composition of the present invention include, but are not limited to, eicosapectaenoic acid, docosahexaenoic acid, and combinations thereof.

Omega-3 fatty acids, at doses from about 1 to 6 g per day, reduce triglycerides, and in particular, reduce the amount of triglycerides within circulating VLDL and LDL. (Agren, et al., Eur. J. Clin. Nutr., Vol. 50, pgs. 765-771 (1996); Sirtori, et al., Atherosclerosis, Vol. 137, pgs. 419-427 (1998).) At low doses, i.e., 1 to 2 grams per day, omega-3 fatty acids provide beneficial effects to the immune system. (Tashiro, et al., Nutrition, Vol. 41, pgs. 551-553 (1998)). Thus, although the scope of the present invention is not to be limited to any theoretical reasoning, Applicant, in order to provide the beneficial effects of omega-3 fatty acids to the immune function, and to maintain an equivalent potency of an anti-hypertriglyceridemic effect, has provided a composition which combines at least one omega-3 fatty acid at a low but effective anti-hypertriglyceridemic dosage with another anti-hypertriglyceridemic agent having an independent mechanism of action.

Pantethine is another antihypertriglyceridemic drug. (Bertolini, et al., Int. J. Clin. Pharmacol. Ther. Toxicol., Vol. 24, pgs. 630-637 (1986); Arsenio, et al., Clin. Ther., Vol. 8, pgs. 537-545 (1986)) with a mechanism of action distinct from that of omega-3 fatty acids, and may be combined with omega-3 fatty acids to maintain an effective anti-hypertriglyceridemic response. Also, pantethine possesses anti-oxidant activity, and has been shown to reduce oxidation of lipoprotein lipids (Bon, et al., Atherosclerosis, Vol. 57, pgs. 99-106 (1985)).

Although Applicant does not intend to be limited to any theoretical reasoning, omega-3 fatty acids are lipids which may be oxidized into an inactive molecule, the combination of omega-3 fatty acids with pantethine may reduce the oxidation, and thus inactivation, of omega-3 fatty acids by the body. As a result, the overall efficacy achieved with omega-3 fatty acids in combination with pantethine may be more than with omega-3 fatty acids alone. Also, pantethine inhibits platelet aggregation. (Hiramatsu, et al., Tokai J. Exp. Clin. Med., Vol. 6, pgs. 49-57 (1981)). Pantethine thus may reduce the likelihood of cardiovascular disease directly by reducing hypertriglyceridemia, oxidized LDL levels, and platelet aggregation, and indirectly by reducing the amount of oxidized (and thus inactivated) omega-3 fatty acids.

LDL can be oxidized by endogenous substances in the blood, such as oxygen, oxygen free radicals, and copper, to induce the formation of conjugated dienes, lipid peroxides, and/or thiobarbituric acid reactive substances.

As mentioned hereinabove, scientific evidence indicates that oxidized LDL is atherogenic, and anti-oxidants capable of reducing the level of oxidized LDL in the body can reduce the risk of cardiovascular disease (Holvoet, 1994; Holvoet, 1998); however, the oxidation of LDL may occur at distinct molecular sites, and the activities of various antioxidants are site-specific. Therefore, it is advantageous to reduce multiple sites of LDL oxidation that lead to cardiovascular disease. Thus, a variety of antioxidants which act at different sites can achieve such a goal. In general, the antioxidants become oxidized by one or more of the endogenous substances mentioned hereinabove. This prevents the oxidation of LDL and thus reduces the level of oxidized LDL.

Thus, in one embodiment, the at least one antioxidant is a carotenoid. Carotenoids inhibit oxidation reactions of LDL which form conjugated dienes and thiobarbituric acid reactive substances. In a preferred embodiment, the carotenoid is lycopene. Lycopene is a natural carotenoid derived from the tomato.

In another embodiment, the at least one antioxidant is Vitamin E, including α- and- γ-tocopherols. Vitamin E, like the carotenoids, inhibits oxidation reactions of LDL which form conjugated dienes and thiobarbituric acid reactive substances.

In another embodiment, the at least one antioxidant is tocotrienol.

In another embodiment, the at least one antioxidant is at least one flavenoid. Flavenoids inhibit oxidation reactions of LDL which form lipid peroxides. Preferably, the at least one flavenoid is selected from the group consisting of catechins, pycnogenol, theaflavins, and combinations thereof. More preferably, the at least one flavenoid is pycnogenol. Pycnogenol is derived from the bark of conifers, such as pine bark, and in particular, the bark of the maritime pine. Pycnogenol is described further in U.S. Pat. No. 5,719,178, issued to Paull, et al.

In another embodiment, the at least one antioxidant is Vitamin C. Vitamin C, like the flavenoids, also inhibits oxidation reactions of LDL which form lipid peroxides. Vitamin C also prevents oxidation of carotenoids and Vitamin E if such substances are included along with Vitamin C in the composition.

In another embodiment, the at least one antioxidant is coenzyme Q10.

In another embodiment, the at least one antioxidant is grape seed extract.

Although the scope of the present invention is not intended to be limited to any theoretical reasoning, it is believed by Applicant that the combination of (i) at least one unsaturated fatty acid, such as at least one omega-3 fatty acid; and at least one of: (ii) pantethine; and (iii) at least one antioxidant selected from those hereinabove described, such as Vitamin E, tocotrienol, lycopene, and/or pycnogenol, collectively and uniquely: (1) preserve the activity of unsaturated fatty acids such as omega-3 fatty acids by preventing their oxidation to an inactive form; (2) allow for a lower daily dosage of unsaturated fatty acids such as omega-3 fatty acids to reduce hypertriglyceridemia (by virtue of the anti-hypertriglyceridemic effect of pantethine), thereby also potentiating the beneficial effects of omega-3 fatty acids on immunity; (3) minimize any effect that unsaturated fatty acids, such as omega-3 fatty acids, may have to increase the amount of oxidized LDL; and (4) interact positively to reduce risk factors associated with atherosclerosis by reducing hypertriglyceridemia, oxidized VLDL, oxidized LDL, and hyper-reactive platelet aggregation, all of which are risk factors for cardiovascular disease, simultaneously. Also, when lycopene is employed as an antioxidant, the at least one unsaturated fatty acid, such as, for example, at least one omega-3 fatty acid, enhances intestinal absorption of the lycopene.

In a preferred embodiment, the composition further comprises an encapsulating medium enclosing the at least one unsaturated fatty acid, and at least one of the pantethine and the at least one antioxidant.

In one embodiment, the encapsulating medium may enclose or encapsulate each of the components of the composition collectively. In another embodiment, the encapsulating medium may enclose or encapsulate each of the components of the composition individually; i.e., there may be separate encapsulated particles of unsaturated fatty acid, and separate encapsulated particles of each of the at least one of the pantethine and the at least one antioxidant.

In another alternative, the encapsulating medium may encapsulate one or more of the components of the composition, individually or collectively, but does not encapsulate all of the components of the composition.

Materials from which the encapsulating medium may be formed include, but are not limited to, ethyl cellulose, hydroxypropylmethyl cellulose, hydroxypropyl cellulose, methyl cellulose, polyvinyl alcohol, polyvinyl acetate butyrate, styrene acrylate copolymers, acrylic acid ester copolymers, and gelatin polymers. Preferably, the encapsulating medium is formed from ethyl cellulose.

The at least one unsaturated fatty acid, and at least one of the pantethine and the at least one antioxidant may be encapsulated with the encapsulating medium into particles by any of a variety of processes known to those skilled in the art. Examples of such processes are described in Lieberman, Pharmaceutical Dosage Forms: Tablets, Vol. 1, Second Edition, pgs. 372-376 (1989); Jayne, Microencapsulation, Processes and Applications, pgs. 103-113 (1973-1974); U.S. Pat. No. 3,300,332, issued to Gorham, et al., and U.S. Pat. No. 5,393,533, issued to Versic.

In one embodiment, the at least one unsaturated fatty acid and at least one of the pantethine and at least one antioxidant are encapsulated into particles having a size of from about 10 microns to about 1,000 microns, preferably from about 10 microns to about 100 microns.

In a more preferred embodiment the composition, in addition to the microparticles herein above described, further comprises a liquid carrier. Most preferably, the liquid carrier includes water. Suitable liquid carriers include, but are not limited to, water, aqueous solutions, and juices, such as fruit juices, including cranberry juice, apple juice, orange juice, and grape juice, and vegetable juices.

In a most preferred embodiment, the at least one unsaturated fatty acid, pantethine, and at least one antioxidant are mixed with the encapsulating medium at a fixed weight ratio of active ingredients (i.e., the at least one unsaturated fatty acid, pantethine, and at least one antioxidant) to encapsulating medium such that the resulting microparticles are isodense with, i.e., having the same density as, the aqueous solution which is the liquid carrier. Thus, there is formed a colloidal suspension of microencapsulated particles of the active ingredients in an aqueous solution that may be administered orally.

The at least one unsaturated fatty acid, pantethine, and at least one antioxidant as hereinabove described are present in amounts effective to reduce plasma triglyceride and cholesterol levels, reduce oxidation of lipoprotein lipids, and reduce platelet aggregation.

In one embodiment, when the composition includes microparticles of the at least one unsaturated fatty acid, pantethine, and the at least one antioxidant, in a liquid carrier, the composition may be administered in an amount which does not exceed 16 ounces, and preferably is from about 4 ounces to about 10 ounces. In such a composition, the at least one unsaturated fatty acid, such as an omega-3 fatty acid, may be present in an amount of from about 0.1 g to about 3.0 g per drink volume, preferably from about 0.2 g to about 2.0 g per drink volume. Pantethine may be present in the composition in an amount of from about 200 mg to about 1,000 mg per drink volume, preferably from about 400 mg to about 900 mg per drink volume.

When Vitamin E is included as an antioxidant in the composition, the Vitamin E may be present in an amount of from about 200 International Units (IU) to about 1,000 IU per drink volume, preferably from about 400 IU to about 800 IU per drink volume.

When lycopene is included as an antioxidant in the composition, the lycopene may be present in an amount of from about 40 mg to about 90 mg per drink volume, preferably from about 55 mg to about 80 mg per drink volume.

When pycnogenol is included as an antioxidant in the composition, the pycnogenol may be present in an amount of from about 50 mg to about 500 mg per drink volume, preferably from about 100 mg to about 300 mg per drink volume.

The invention now will be described with respect to the following examples; however, the scope of the present invention is not intended to be limited thereby.

### EXAMPLE 1

A formulation was prepared by adding the following ingredients to a fruit drink (guava juice: water, guava puree, fruit juice concentrate containing 22% fruit juice).

1. Approximately 5 grams microencapsulated fish oil concentrate (with lemon flavor) containing approximately 350 mg of eicosapectaenoic acid and docosahexaenoic acid.

2. Approximately 1.5 grams of microencapsulated 50% pantethine powder.

3. Approximately 400 I.U. of powdered vitamin E.

4. Approximately 1 gram of powdered vitamin C.

5. Approximately 1 gram of bioflavenoids (400 mg Citrus Bioflavenoids [37% Hesperidin], 200 mg Rose Hips Powder [Rosae pseudofructus], and 200 mg Acerola powder)

6. Approximately 50 mg of powdered grape seed extract.

The formulated drink provides a mixture of components that a) may reduce high triglyceride levels, platelet hyper-aggregation, and oxidized low density lipoprotein levels, b) is palatable (by virtue of microencapsualtion of ingredients into a fruit drink) and c) is simple to prepare.

Thus, this formulation provides a simple, palatable drink which includes active components at appropriate dosages to reduce elevated plasma triglyceride and oxidized LDL levels simultaneously, as well as platelet hyper-aggregation.

### EXAMPLE 2

Obese female C57BL/6J mice (ob/ob) are known to be hyperlipidemic, with much higher plasma triglyceride and cholesterol levels than their lean littermates. Such ob/ob mice were divided into 2 groups and fed Hartz guinea pig chow (18% protein, 4% fat, 15% ash fiber with 200 mg/kg vitamin C and 40 I.U./kg vitamin E) with (test group) or without (control group) the addition of vitamin E (20 I.U./day), vitamin C (25 mg/day), bioflavenoids (Citrus Bioflavenoids [37% Hesperidin], Rose Hips Powder [Rosae pseudofructus], and Acerola powder; 20 mg/day), pantethine (40 mg/day), and eicosapectaenoic acid plus docosahexaenoic acid from fish oil (25-50 mg/day) daily for 19 days. Lean littermates (+/?) were similarly treated with Hartz guinea pig diet alone. Obese mice consumed about 4 grams of food per day and lean mice consumed about 3 grams per day. After 19 days of treatment, mice were sacrificed at 4-6 hours after light onset on the 20th day of the study. Serum was collected for the analyses of serum triglyceride and total cholesterol levels.

The mean serum levels±S.E.M. of triglyceride and cholesterol for the three groups were as follows.

| SERUM | CONTROL GROUP | TEST GROUP | LEAN GROUP |
|---|---|---|---|
| PARAMETER | (ob/ob) | (ob/ob) | (+/?) |
| Triglyceride | 232 +/− 30 mg/dl | 68 +/− 6 mg/dl* | 40 +/− 2 mg/dl |
| Cholesterol | 83 +/− 6 mg/dl | 54 +/− 5 mg/dl* | 65 +/− 2 mg/dl |

An asterisk denotes a significant change from the control group (P < 0.05).

The addition of a formulation in accordance with the present invention to the diet of hyperlipidemic mice reduced the serum triglyceride level by 70% and the serum total cholesterol level by 34% to levels observed in normal lean mice.

The disclosure of all patents and publications, including published patent applications, are herein incorporated by reference to the same extent as if patent and publication specifically and individually were incorporated by reference.

It is to be understood, however, that the scope of the present invention is not to be limited to the specific embodiments described above. The invention may be practiced other than as particularly described and still be within the scope of the accompanying claims.

What is claimed is:

1. A composition, comprising:
(a) at least one unsaturated fatty acid; and at least one of:
(b) pantethine; and (c) at least one antioxidant selected from the group consisting of Vitamin C, Vitamin E, tocotrienol, carotenoids, flavenoids, coenzyme Q10, and grape seed extract.

2. The composition of claim 1 wherein said at least one unsaturated fatty acid is selected from the group consisting of omega-3 fatty acids, α-linolenic acid, and oleic acid.

3. The composition of claim 2 wherein said at least one unsaturated fatty acid is at least one omega-3 fatty acid.

4. The composition of claim 3 wherein said at least one omega-3 fatty acid is selected from the group consisting of eicosapectaenoic acid, docosahexaenoic acid and combinations thereof.

5. The composition of claim 1 wherein said at least one antioxidant is Vitamin C.

6. The composition of claim 1 wherein said at least one antioxidant is Vitamin E.

7. The composition of claim 1 wherein said at least one antioxidant is tocotrienol.

8. The composition of claim 1 wherein said at least one antioxidant is a carotenoid.

9. The composition of claim 8 wherein said carotenoid is lycopene.

10. The composition of claim 1 wherein said at least one antioxidant is a flavenoid.

11. The composition of claim 10 wherein said flavenoid is selected from the group consisting of pycnogenol, catechins, theaflavins, and combinations thereof.

12. The composition of claim 11 wherein said flavenoid is pycnogenol.

13. The composition of claim 1 wherein said at least one antioxidant is coenzyme Q10.

14. The composition of claim 1 wherein said at least one antioxidant is grape seed extract.

15. The composition of claim 1, and further comprising:
(d) an encapsulating medium enclosing said at least one unsaturated fatty acid, and at least one of said pantethine and said at least one antioxidant.

16. The composition of claim 15 wherein said encapsulating medium is formed from a material selected from the group consisting of ethyl cellulose, hydroxypropylmethyl cellulose, hydroxypropyl cellulose, methyl cellulose, polyvinyl alcohol, polyvinyl acetate butyrate, styrene acrylate copolymers, acrylic acid ester copolymers, and gelatin polymers.

17. The composition of claim 16 wherein said encapsulating medium is formed from ethyl cellulose.

18. The composition of claim 15 and further comprising a liquid carrier.

19. The composition of claim 18 wherein said encapsulating medium is isodense with said liquid carrier.

20. The composition of claim 18 wherein said liquid carrier includes water.
